(19) United States
(12) Patent Application Publication
(10) Pub. No.: US 2008/0119563 A1
(43) Pub. Date: May 22, 2008

(54) METHODS FOR RNA DESILYLATION

(75) Inventors: Andrei Laikhter, Iowa City, IA (US); William Martin, Coralville, IA (US); Erin Edgar, Iowa City, IA (US)

Correspondence Address: JOHN PETRAVICH;INTEGRATED DNA TECHNOLOGIES, INC., 8180 MCCORMICK BLVD., SKOKIE, IL, 60076-2920, US

(73) Assignee: INTEGRATED DNA TECHNOLOGIES, INC., Skokie, IL (US)

(21) Appl. No.: 11/940,516

(22) Filed: Nov. 15, 2007

Related U.S. Application Data
(60) Provisional application No. 60/866,469, filed on Nov. 20, 2006.

(51) Int. Cl. A61K 31/14; C07H 21/00

(52) U.S. Cl. 514/642; 536/25.31

(57) ABSTRACT

The invention provides tetraalkyl ammonium fluoride derivatives or a pyridine hydrogen fluoride complex to remove silyl protecting groups in oligoribonucleotides synthesis.

### CROSS-REFERENCE TO RELATED APPLICATION

The present application claims priority under 35 U.S.C. § 119(e) to U.S. provisional patent application No. 60/866,469 filed 20 Nov. 2006. The entire teachings of the above application are incorporated herein by reference.

### FIELD OF THE INVENTION

This invention pertains to methods of ribonucleic acid (RNA) synthesis, specifically, new methods for the removal of the 2′-OH protecting groups during the synthesis of oligoribonucleotides.

### BACKGROUND OF THE INVENTION

RNA or segments of RNA are vital tools in current scientific applications. RNA can be used to study cellular processes, or they can be used to inhibit gene expression. The methods of synthesis of oligoribonucleotides have paralleled the methods of synthesis of deoxyribonucleic acid (DNA), but RNA synthesis has traditionally been more burdensome due to the 2′ hydroxyl group present in RNA. The 5′ hydroxyl group and the 2′ position need to be protected during synthesis, but each position's protecting group needs to be removed at different times. This has led to more complex synthesis methods for RNA synthesis. One example of RNA synthesis is described by Ogilvie et al. (Proc. Natl. Acad. Sci., Vol. 85, pp. 5764-5768, August 1988).

In Ogilvie et al., the 2′-hydroxyl groups are protected by tert-butyl-dimethyl silyl (TBDMS) protecting groups. Typically, the synthesized RNA is deprotected (the silyl protecting groups are removed) after coupling using tetrabutyl ammonium fluoride (TBAF) in tetrahydrofuran (see Glen Research Report, Vol. 4, No. 1, March 1991, RNA Synthesis—Problems in Deprotection). This method of deprotection can take hours and, particularly with longer oligoribonucleotides, will not work completely, leaving a protecting group that may inhibit the usefulness of the resulting RNA.

Another alternative is to use an alternative 5′ protecting group instead of the traditional dimethoxytrityl (DMT) group (see Scaringe et al., U.S. Pat. No. 5,889,136). In Scaringe et al., a silyl ether group is used at the 5′ position, and the 2′ protecting group is 2′-O-bis(2-acetoxyethoxy)methyl (ACE) orthoester. This method requires extensive cycle and reagent changes that increase the complexity of the synthesis.

Another alternative is to substitute the 2′TBDMS group with 2′-O-triisopropylsilyloxymethyl (TOM). The structure of TOM is sterically favorable and results in better coupling yields under mild conditions. It is stable in basic or weakly acidic conditions. However, TOM is not favorable under certain conditions such as heating, and results in side products that are usually neutral.

In Duplaa et al., (U.S. Pat. No. 5,552,539) triethylamine trihydrofluoride (TEA/3HF) is used, typically in an organic solvent such as acetonitrile, to remove the protecting groups, particularly TBDMS. TEA/3HF takes less time (about 2-20 hours), can be used with longer oligonucleotides and offers a more complete deprotection.

Although TEA/3HF is an improvement over prior deprotection reagents, there is a need to provide alternative deprotection reagents, particularly reagents that can provide faster deprotection times under mild conditions. Additionally, there is a need for reagents that allow the base deprotection and desilylation to occur while the oligonucleotide remains on the support.

The proposed method provides alternative reagents, including tetraalkyl ammonium fluoride derivatives and pyridine hydro fluoride, which remove silyl protecting groups in less than two hours under mild conditions.

### BRIEF SUMMARY OF THE INVENTION

The proposed method provides alternative reagents, including tetraalkyl ammonium fluoride derivatives and pyridine hydro fluoride, which remove silyl protecting groups in less than two hours under mild conditions. In one group of embodiments, the reagents can be used while before the oligonucleotide is removed from the support.

### BRIEF DESCRIPTION OF THE DRAWINGS

FIG. 1 is an ESI mass spectroscopy trace of SEQ ID NO.1 that was synthesized using tetraethylammonium fluoride as the deprotecting reagent.

FIG. 2 is an ESI mass spectroscopy trace of SEQ ID NO.2 that was synthesized using tetraethylammonium fluoride as the deprotecting reagent.

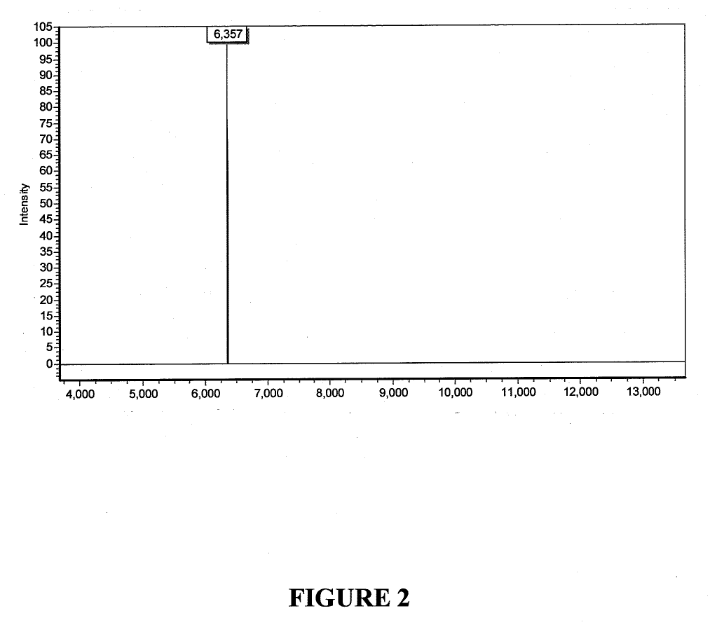

### DETAILED DESCRIPTION OF THE INVENTION

The proposed method provides alternative reagents, including tetraalkyl ammonium fluoride derivatives and pyridine hydro fluoride, which remove silyl protecting groups in less than two hours under mild conditions. In one embodiment, the desilylation can occur while the oligonucleotide is still attached to the support. The proposed deprotection reagents can be used with RNA synthesis procedures well known in the art, such as those described in Duplaa et al. In one embodiment, tetraethylammonium fluoride in dimethly sulfoxide (DMSO) solution is used to remove silyl protecting groups. In another embodiment, a DMSO/pyridine/hydrogen fluoride pyridine solution is used to remove silyl groups in otherwise conventional RNA synthesis conditions. In another embodiment, the proposed deprotecting reagents can be used to remove silyl groups in less than two hours. In another embodiment, the proposed deprotecting reagents can be removed at room temperature using sonication.

The term “oligonucleotides” refers to synthesized RNA or DNA polymers, and “oligoribonucleotides” would be a subset of “oligonucleotides” that comprise at least one ribonucleotide monomer. One or more of the DNA and RNA monomers can be modified with a label, linking group or other modifications known in the art.

The following examples further illustrate the invention but, of course, should not be construed as in any way limiting its scope.

### EXAMPLE 1

This example demonstrates oligonucleotide synthesis and desilylation using tetraethylammonium fluoride.

Two oligonucleotides were synthesized using 2′-TBDMS protected standard RNA phosphoramidite chemistry on an Applied Biosystems Model Expedite 8909 DNA/RNA synthesizer. Reactions were done on a 1 umole scale.

| | |
|---|---|
| SEQ ID NO: 1: | 5′ aTTTTTTTTTTTTTTT 3′ |
| SEQ ID NO: 2: | 5′ gaacuucaggcuccugggcT 3′ |

### T=deoxythymidine (DNA), u=uridine, a=adenosine, g=guanine, c=cytosine, (RNA).

Following synthesis, the controlled pore glass (CPG) solid support was transferred to a 2 ml microfuge tube. Oligonucleotides were cleaved from the CPG and deprotected by incubation for 30 minutes at 65° C. in 1 ml of 40% methylamine solution in water. The supernatant was removed and supernatants were pooled and dried. The t-butyl-dimethylsilyl protecting group was removed from the RNA residue by treatment with 500 μL of 15% solution of tetraethylammonium fluoride in DMSO at room temperature in an ultrasonic bath for 30 minutes. The oligonucleotide was precipitated by 1.5 ml of n-butanol; the sample was cooled at −70° C. for 1 hour and then centrifuged at 10,000 g for 10 minutes. The supernatant was decanted, and the pellet was washed with n-butanol one more time.

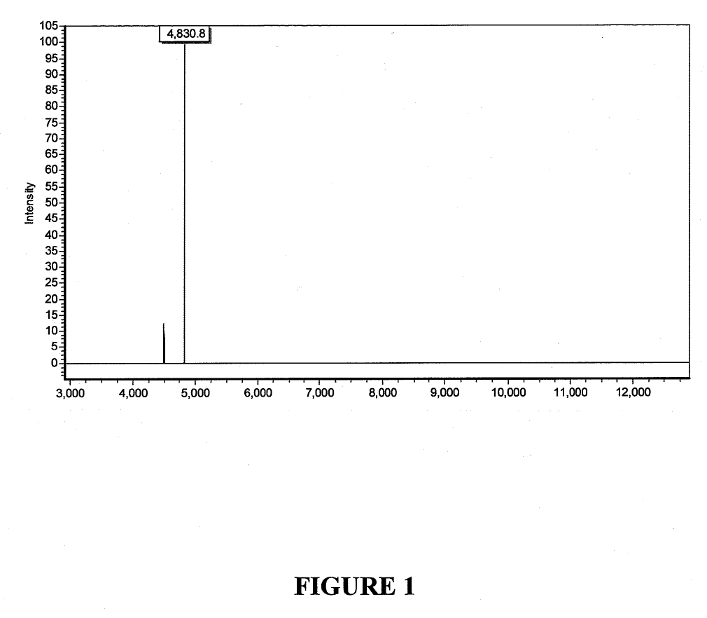

The compound identity was verified after synthesis and purification by ESI mass spectroscopy. Mass traces are shown in FIG. 1. Measured mass for Substrate SEQ ID NO:1 was 4830.8 (calculated mass 4830.2). Measured mass for Substrate SEQ ID NO:2 was 6357.0 (calculated mass 6356.9).

### EXAMPLE 2

The following example demonstrates the synthesis of oligonucleotides using pyridine hydrofluoride as the desilylation reagent.

Oligonucleotides SEQ ID NO:1 and SEQ ID NO:2 have been synthesized and cleaved from CPG as described above in Example 1. The t-butyl-dimethylsilyl protecting group was removed from the RNA residue by treatment with 500 μL of solution 1:2 (v/v) of pyridine hydrofluoride (HF)/pyridine (Pyr) at room temperature in an ultrasonic bath for 30 minutes. Final product was isolated and analyzed as described in Example 1. The ratio of HF to Pyr in Olah reagent is 9:1 (70% HF, 30% Pyr), but the protecting group was successfully removed using HF/Pyr ratios between 6:1 to 1:1. In one embodiment, the ratio is 3:1 that corresponds to a 1:2 ratio Olah/Pyr.

### EXAMPLE 3

The following example demonstrates the synthesis and desilylation using pyridine hydrofluoride on a polystyrene solid support.

An oligonucleotide of SEQ ID NO:2 was synthesized using 2′-TBDMS protected standard RNA phosphoramidite chemistry on an Applied Biosystems Model Expedite 8909 DNA/RNA synthesizer. Reactions were done on the 1 umole scale.

Following synthesis, the polystyrene (PS) solid support was transferred to a 2 ml microfuge tube. Oligonucleotide was cleaved and deprotected by incubation for 60 minutes at 55° C. in 1 ml of neat propylamine without detaching the oligonucleotide from the solid support. Excess of propylamine was removed and solid support was washed with 1 mL of THF.

The t-butyl-dimethylsilyl protecting group was removed from the RNA residue by treatment with 500 μL of solution 1:2:3 (v/v) of pyridine hydrofluoride/pyridine/THF at 40° C. for 30 minutes. Solid support was washed with 2×1 mL portions of butanol. The oligonucleotide was eluted with 1.5 mL of the solution containing 20% of methanol in DI water.

The compound identity was verified after synthesis and purification by ESI mass spectroscopy. The measured mass for Substrate SEQ ID NO:2 was 6357.0 (calculated mass 6356.9).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition for deprotecting 2′-hydroxyls of protected RNA molecules, the composition comprising tetraalkylammonium fluoride or pyridine hydrogen fluoride.

2. The composition of claim 1 wherein the tetraalkylammonium fluoride is tetraethylammonium fluoride.

3. The composition of claim 2 wherein the tetraethylammonium fluoride is in a mixture containing dimethyl sulfoxide.

4. The composition of claim 1 wherein the composition comprises pyridine hydrogen fluoride in a mixture containing Olah reagent.

5. The composition of claim 1 wherein the tetraalkylammonium fluoride or pyridine hydrogen fluoride are removing silyl protecting groups.

6. The composition of claim 5 wherein the silyl protecting group is tert-butyl-dimethyl silyl or 2′-O-triisopropylsilyloxymethyl.

7. The composition of claim 1 wherein the tetraalkylammonium fluoride or pyridine hydrogen fluoride are removing a 2′-O-bis(2-acetoxyethoxy)methyl orthoester protecting group.

8. A method for deprotecting 2′-hydroxyls of protected RNA molecules, the method comprising:
a) combining protected RNA with the composition from claim 1;
b) applying heat or sonication to the RNA and the composition from claim 1 for about 30-180 minutes.

9. The method of claim 8 wherein the heat or sonication are applied for about 30 to 120 minutes.

10. The method of claim 8 wherein the claim 1 composition is tetraethylammonium fluoride in dimethyl sulfoxide.

11. A method for recovering synthesized oligonucleotides from a solid support, the method comprising the steps of; (1) providing a solid synthetic support having synthesized oligonucleotides bound thereto, and (2) incubating the solid support with an amine reagent under conditions suitable to cleave and deprotect the oligonucleotide, (3) removing the amine reagent in a manner that substantially removes free residues of cleaved protecting groups and permits the cleaved and deprotected oligonucleotide to be preferentially retained on the support, and (4) incubating the solid support with a desilylation reagent, said desilylation reagent comprising pyridine hydrogen fluoride and Olah reagent, under conditions suitable to remove silyl protecting groups while leaving the oligonucleotide on the support.

12. The method of claim 11 wherein the amine reagent is a neat amine reagent.

13. The method of claim 12 wherein the neat amine reagent is a neat propylamine reagent.

14. A method of removing base protecting groups and 2′ protecting groups positions of an oligonucleotide bound to a support, the method comprising:
a) introducing an neat propylamine reagent to the oligonucleotide bound to the support to cleave base protecting groups from the oligonucleotide;
b) introducing a reagent comprising pyridine hydrogen fluoride and Olah reagent to the oligonucleotide bound to the support to remove 2′ protecting groups.

15. The method of claim 14 wherein the amine reagent is removed from the oligonucleotide bound to the support before introducing the desilylation reagent.

16. The method of claim 15 wherein the amine reagent is removed by a vacuum, gravitational pressure or through a tetrahydrofuran rinse step.


SEQUENCE LISTING (2 sequences)

SEQ ID NO: 1 - DNA, 16 bp, Artificial
  Synthesized oligonucleotide
attttttttttttttt

SEQ ID NO: 2 - DNA, 20 bp, Artificial
  Synthesized oligonucleotide
gaacuucaggcuccugggct